(19) United States
(12) Patent Application Publication
(10) Pub. No.: US 2025/0039618 A1
(43) Pub. Date: Jan. 30, 2025

(54) SYSTEM AND METHOD FOR DETERMINING MAGNETIC RETENTION FORCE FOR MEDICAL DEVICE

(71) Applicant: Cochlear Limited, Macquarie University, NSW (AU)

(72) Inventors: Nathan Isaacson, Balgowlah (AU); Peter John Russell, Regentville (AU); Padraig Hurley, Frenches Forest (AU); Sabina Dabkowski-Chandler, Camperdown (AU); Lily Henson, Haberfield (AU)

(21) Appl. No.: 18/713,863

(22) PCT Filed: Dec. 1, 2022
(86) PCT No.: PCT/IB2022/061671

§ 371 Date: May 28, 2024

Related U.S. Application Data
(60) Provisional application No. 63/268,833, filed on Mar. 3, 2022.
(60) Provisional application No. 63/266,652, filed on Jan. 11, 2022.

(51) Int. Cl. H04R 25/00; A61N 1/05

(52) U.S. Cl. CPC H04R 25/60; A61N 1/0541; H04R 25/65; H04R 2225/67

(57) ABSTRACT

An apparatus includes a housing having an external surface configured to be placed on a non-magnetic material over-laying a first device comprising a first ferromagnetic or ferrimagnetic material. The apparatus further includes a second ferromagnetic or ferrimagnetic material and/or an electromagnet within the housing. The second ferromagnetic or ferrimagnetic material and/or the electromagnet is configured to generate an attractive magnetic force with the first device. The apparatus is configured to controllably adjust an attractive magnetic force between the first device and the second ferromagnetic or ferrimagnetic material and/or the electromagnet along a longitudinal axis substantially perpendicular to the surface.

### BACKGROUND

### Field

The present application relates generally to systems and methods for affixing an external component to a recipient's body using magnetic attraction between the external component and a device implanted on or within a recipient's body.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a housing having an external surface configured to be placed on a non-magnetic material overlaying a first device comprising a first ferromagnetic or ferrimagnetic material. The apparatus further comprises a second ferromagnetic or ferrimagnetic material within the housing. The second ferromagnetic or ferrimagnetic material is configured to generate an attractive magnetic force with the first device. The apparatus further comprises a movable coupler in mechanical communication with the housing and the second ferromagnetic or ferrimagnetic material. The coupler is configured to controllably adjust a distance between the second ferromagnetic or ferrimagnetic material and the first device along a longitudinal axis substantially perpendicular to the surface.

In another aspect disclosed herein, an apparatus comprises a housing configured to be placed over a non-magnetic material overlaying a device comprising a first ferromagnetic or ferrimagnetic material. The apparatus further comprises a second ferromagnetic or ferrimagnetic material within the housing. The second ferromagnetic or ferrimagnetic material is configured to generate an attractive magnetic force with the device. The apparatus further comprises a gauge in mechanical communication with the housing and the second ferromagnetic or ferrimagnetic material. The gauge is configured to generate an indication of a magnitude of the attractive magnetic force along a direction towards the device and/or a thickness of the non-magnetic material between the device and the housing.

In another aspect disclosed herein, an apparatus comprises a housing having an external surface configured to be placed on a non-magnetic material overlaying a first device comprising a first ferromagnetic or ferrimagnetic material. The apparatus further comprises an electromagnet within the housing. The electromagnet is configured to generate and controllably adjust an attractive magnetic force between the electromagnet and the first device along a longitudinal axis substantially perpendicular to the external surface. The attractive magnetic force approximates a magnetic force of a predetermined magnetic configuration of an external second device configured to overlay the first device in place of the apparatus.

In another aspect disclosed herein, an apparatus comprises a housing having an external surface configured to be placed on a non-magnetic material overlaying a first device comprising a first ferromagnetic or ferrimagnetic material. The apparatus further comprises a cavity within the housing and a permanent magnet within the cavity. The permanent magnet is configured to move within the cavity along a longitudinal axis substantially perpendicular to the external surface. The permanent magnet is configured to generate an attractive magnetic force with the first device. The apparatus further comprises an electromagnet within the housing and configured to generate and controllably adjust a retention attractive magnetic force between the electromagnet and the permanent magnet.

In another aspect disclosed herein, a method comprises placing an apparatus on a recipient's body over a portion of tissue overlaying an implanted device. The apparatus comprises a ferromagnetic or ferrimagnetic material configured to generate an attractive magnetic force with the implanted device. The method further comprises, while the apparatus is on the recipient's body, using the apparatus to generate information indicative of a magnitude of the attractive magnetic force and/or indicative of a separation between the apparatus and the implanted device. The method further comprises removing the apparatus from the recipient's body. The method further comprises, in response to the information, selecting a magnetic configuration for an external device. The method further comprises placing the external device having the selected magnetic configuration over the portion of tissue such that the external device is magnetically held on the recipient's body.

In another aspect disclosed herein, a method comprises placing an apparatus on a recipient's body over a portion of tissue overlaying an implanted device. The method further comprises, while the apparatus is on the recipient's body, generating information indicative of a magnitude of an attractive magnetic force between the apparatus and the implanted device and/or indicative of a separation distance between the apparatus and the implanted device. The method further comprises, in response to the information, selecting a magnetic configuration for the apparatus. The method further comprises modifying the apparatus to have the selected magnetic configuration.

### BRIEF DESCRIPTION OF THE DRAWINGS

Implementations are described herein in conjunction with the accompanying drawings, in which:

FIG. 1A is a perspective view of an example cochlear implant auditory prosthesis implanted in a recipient in accordance with certain implementations described herein;

FIG. 1B is a perspective view of an example fully implantable middle ear implant auditory prosthesis implanted in a recipient in accordance with certain implementations described herein;

FIG. 1C schematically illustrates a side cross-sectional view of an example transcutaneous system comprising an implantable component and an external component;

FIG. 2 schematically illustrates a cross-sectional view of an example apparatus in accordance with certain implementations described herein;

FIGS. 3A and 3B schematically illustrate a cross-sectional view and a top view, respectively, of an example apparatus having an example movable coupler in accordance with certain implementations described herein;

FIGS. 3C-3E schematically illustrate perspective views of the example apparatus of FIGS. 3A-3B in three different configurations with the coupler at various positions and/or orientations relative to the housing in accordance with certain implementations described herein;

FIG. 4 schematically illustrates a cross-sectional view of an example apparatus having a coupler configured to be controllably and linearly slid along the longitudinal axis relative to the housing in accordance with certain implementations described herein;

FIG. 5 schematically illustrates a cross-sectional view of an example apparatus comprising a gauge in accordance with certain implementations described herein

FIG. 6 schematically illustrates a side cross-sectional view and a top cross-sectional view of another example apparatus comprising a gauge in accordance with certain implementations described herein;

FIGS. 7A and 7B schematically illustrate cross-sectional views of still another example apparatus comprising a gauge in accordance with certain implementations described herein;

FIG. 8 schematically illustrates a cross-sectional view of an example apparatus comprising an electromagnet in accordance with certain implementations described herein;

FIG. 9 schematically illustrates a cross-sectional view of another example apparatus comprising an electromagnet in accordance with certain implementations described herein;

FIG. 10 is a flow diagram of an example method in accordance with certain implementations described herein; and

FIG. 11 is a flow diagram of an example method in accordance with certain implementations described herein.

### DETAILED DESCRIPTION

Certain implementations described herein provide an apparatus for providing information relevant for determining whether a particular implant recipient can be supported by at least one of the different retention magnet options that are available for a particular external device (e.g., cochlear implant sound processor) to be used with and magnetically retained by the recipient's implant (e.g., cochlear implant). The apparatus can provide information relevant for determining the appropriate external device magnet configuration for a particular patient and/or to estimate a skin flap thickness (SFT) of the recipient's tissue between the implant and the external device. In certain implementations, the apparatus is controllably adjustable (e.g., includes a movable magnet and/or an electromagnet with a controllably adjustable magnetic field strength) to adjust and/or be responsive to the attractive magnetic force with the underlying implant. In certain other implementations, the apparatus comprises a magnet (e.g., permanent magnet; electromagnet) in mechanical communication with a force gauge configured to generate an indication of the attractive magnetic force between the magnet and the underlying implant.

The teachings detailed herein are applicable, in at least some implementations, to any type of implantable or non-implantable stimulation system or device (e.g., implantable or non-implantable auditory prosthesis device or system) configured to provide stimulation signals and/or medicament dosages to a portion of the recipient's body in response to received information and/or control signals (e.g., implantable sensor prostheses; implantable stimulation system; implantable medicament administration system) from an external portion of the system or device and/or any type of implantable sensor system configured to provide sensor signals from an implanted portion of the system or device to an external portion of the system or device. Implementations can include any type of medical device that can utilize the teachings detailed herein and/or variations thereof. Furthermore, while certain implementations are described herein in the context of auditory prosthesis devices, certain other implementations are compatible in the context of other types of devices or systems that provide a wide range of therapeutic benefits to recipients, patients, or other users. For example, other sensory prosthesis systems that are configured to evoke other types of neural or sensory (e.g., sight, tactile, smell, taste) percepts are compatible with certain implementations described herein, including but are not limited to: vestibular devices (e.g., vestibular implants), visual devices (e.g., bionic eyes), visual prostheses (e.g., retinal implants), somatosensory implants, and chemosensory implants. In some implementations, the teachings detailed herein and/or variations thereof can be utilized in other types of implantable medical devices beyond sensory prostheses. For example, apparatus and methods disclosed herein and/or variations thereof can be used with one or more of the following: sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; pain relief devices; etc. Implementations can include any type of medical system that can utilize the teachings detailed herein and/or variations thereof.

Merely for ease of description, apparatus and methods disclosed herein are primarily described with reference to an illustrative medical device, namely an implantable transducer assembly including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses. In some implementations, the teachings detailed herein and/or variations thereof can be utilized in other types of prostheses beyond auditory prostheses.

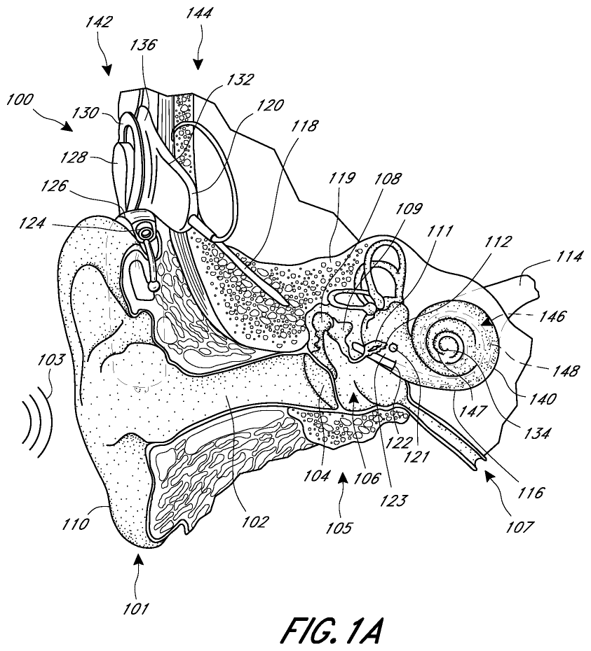

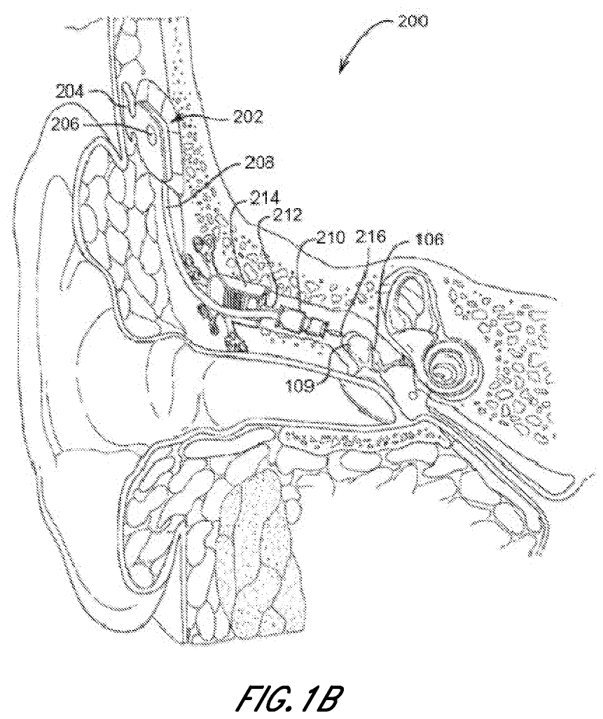

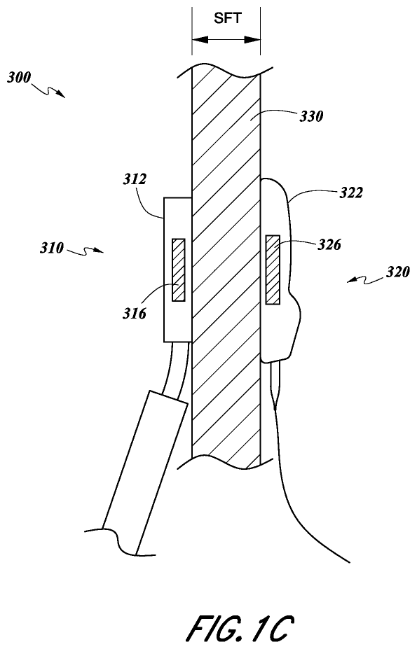

FIG. 1A is a perspective view of an example cochlear implant auditory prosthesis 100 implanted in a recipient in accordance with certain implementations described herein. The example auditory prosthesis 100 is shown in FIG. IA as comprising an implanted stimulator unit 120 and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1A with a subcutaneously implantable microphone assembly, as described more fully herein. In certain implementations, the example cochlear implant auditory prosthesis 100 of FIG. 1A can be in conjunction with a reservoir of liquid medicament as described herein.

As shown in FIG. 1A, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1A, the example auditory prosthesis 100 comprises one or more components which are temporarily or permanently implanted in the recipient. The example auditory prosthesis 100 is shown in FIG. 1A with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1A, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1A schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

FIG. 1B schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 1B comprises a biocompatible implantable assembly 202 (e.g., comprising an implantable capsule) located subcutaneously (e.g., beneath the recipient's skin and on a recipient's skull). While FIG. 1B schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer 206 (e.g., a microphone comprising a diaphragm and an electret or piezoelectric transducer) that is positioned to receive acoustic signals through the recipient's overlying tissue. The implantable assembly 202 may further be utilized to house a number of components of the fully implantable auditory prosthesis 200. For example, the implantable assembly 202 can include an energy storage device and a signal processor (e.g., a sound processing unit). Various additional processing logic and/or circuitry components can also be included in the implantable assembly 202 as a matter of design choice.

For the example auditory prosthesis 200 shown in FIG. 1B, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). In certain implementations, the example auditory prosthesis 100, 200 shown in FIGS. 1A and 1B can comprise an implantable microphone assembly, such as the microphone assembly 206 shown in FIG. 1B. For such an example auditory prosthesis 100, the signal processor of the implantable assembly 202 can be in operative communication (e.g., electrically interconnected via a wire) with the microphone assembly 206 and the stimulator unit of the main implantable component 120. In certain implementations, at least one of the microphone assembly 206 and the signal processor (e.g., a sound processing unit) is implanted on or within the recipient.

The actuator 210 of the example auditory prosthesis 200 shown in FIG. 1B is supportably connected to a positioning system 212, which in turn, is connected to a bone anchor 214 mounted within the recipient's mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient's tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient's auditory system. To compensate for the decreased acoustic signal strength reaching the microphone assembly 202 by virtue of being implanted, the diaphragm of an implantable microphone assembly 202 can be configured to provide higher sensitivity than are external non-implantable microphone assemblies. For example, the diaphragm of an implantable microphone assembly 202 can be configured to be more robust and/or larger than diaphragms for external non-implantable microphone assemblies.

The example auditory prostheses 100 shown in FIG. 1A utilizes an external microphone 124 and the auditory prosthesis 200 shown in FIG. 1B utilizes an implantable microphone assembly 206 comprising a subcutaneously implantable acoustic transducer. In certain implementations described herein, the auditory prosthesis 100 utilizes one or more implanted microphone assemblies on or within the recipient. In certain implementations described herein, the auditory prosthesis 200 utilizes one or more microphone assemblies that are positioned external to the recipient and/or that are implanted on or within the recipient, and utilizes one or more acoustic transducers (e.g., actuator 210) that are implanted on or within the recipient. In certain implementations, an external microphone assembly can be used to supplement an implantable microphone assembly of the auditory prosthesis 100, 200. Thus, the teachings detailed herein and/or variations thereof can be utilized with any type of external or implantable microphone arrangement, and the acoustic transducers shown in FIGS. 1A and 1B are merely illustrative.

FIG. 1C schematically illustrates a side cross-sectional view of an example transcutaneous system 300 comprising an implantable component 310 and an external component 320. For example, the transcutaneous system 300 can comprise an auditory prosthesis 100, 200 in which the implantable component 310 comprises one or more active elements (e.g., stimulator unit 120; assembly 202; vibrating actuator; not shown in FIG. 1C) configured to deliver stimuli to the recipient's body and the external component 320 (e.g., external component 142) can comprise components worn outside the recipient's body (e.g., on the recipient's body) and configured to operate with the implantable component 310 (e.g., external microphone 124; sound processing unit 126; a power source; external transmitter unit 128).

The implantable component 310 can comprise at least one implantable housing 312 configured to be positioned beneath tissue 330 of the recipient's body (e.g., beneath the skin, fat, and/or muscular layers) and above a bone (e.g., skull) in a portion of the recipient's body (e.g., the head). The at least one implantable housing 312 can contain at least one internal communication coil (not shown; e.g., a planar electrically conductive wire with multiple windings; multiple turns of electrically insulated single-strand or multi-strand platinum or gold wire having a substantially circular, rectangular, spiral, or oval shape or other shape) and at least one internal magnetic (e.g., ferromagnetic; ferrimagnetic; permanent magnet) material 316 (e.g., disk; plate). The at least one internal magnetic material 316 can be configured to establish a magnetic attraction between the external component 320 and the implantable component 310 sufficient to hold the external component 320 against an outer surface of the tissue 330. The at least one implantable housing 312 can comprise a first portion configured to contain the at least one internal magnetic material 316 and the at least one internal communication coil and a second portion configured to contain the one or more active elements, or the at least one implantable housing 312 can comprise a single housing portion configured to contain the at least one internal magnetic material 316, the at least one internal communication coil, and the one or more active elements.

The external component 320 can comprise an external housing 322 configured to be positioned on an outer surface of the tissue 330 (e.g., skin) and contains at least one external communication coil (not shown; e.g., a planar electrically conductive wire with multiple windings) and at least one external magnetic (e.g., ferromagnetic; ferrimagnetic; permanent magnet) material 326 (e.g., disk; plate). The at least one external communication coil can be configured to be in wireless electrical communication (e.g., wirelessly receiving and/or transmitting data and/or control signals and/or wirelessly transmitting power to the implantable component 310 via at least one radio-frequency link and/or inductive coupling) with the at least one internal communication coil when the external component 320 is positioned on the tissue 330 of the recipient above the internal component 310. The at least one external magnetic material 326 can be configured to establish a magnetic attraction between the external component 320 and the implantable component 310 sufficient to hold the external component 320 against the outer surface of the tissue 330.

The coupling coefficient between the at least one internal communication coil and the at least one external communication coil is inversely dependent on the distance between the at least one internal communication coil and the at least one external communication coil. In addition, the strength of the magnetic attraction between the at least one internal magnetic material 316 and the at least one external magnetic material 326 is inversely dependent on the distance between the at least one internal magnetic material 316 and the at least one external magnetic material 326. To provide a sufficiently large coupling coefficient, the at least one external communication coil can be configured to be as close as possible to the at least one internal communication coil (e.g., a distance as close as possible to the thickness of the tissue 330 between the at least one implantable housing 312 and the external housing 322). To produce a sufficiently strong magnetic attraction, the at least one external magnetic material 326 can be configured to be as close as possible to the at least one internal magnetic material 316 (e.g., a distance as close as possible to the thickness of the tissue 330 between the at least one implantable housing 312 and the external housing 322).

The thickness of the tissue 330 between the at least one implantable housing 312 and the external housing 322 can be referred to as the skin flap thickness (SFT), as denoted in FIG. 1C. Depending on the recipient, the SFT for auditory prosthesis systems can be, for example, in a range of 2 millimeters to 12 millimeters, and the SFT for other types of systems can have a larger maximum value. In addition to differences of SFT among different recipients, the SFT can also change under various physiological situations (e.g., weight loss or gain by recipient; growth of the recipient). Information regarding the SFT of the recipient and/or the strength of the magnetic attraction achievable using the implantable component 310 of the recipient and potentially available external components 320 and/or external magnetic materials 326 can be helpful for selecting an appropriate external component 320 and/or at least one external magnetic material 326 for the transcutaneous system 300.

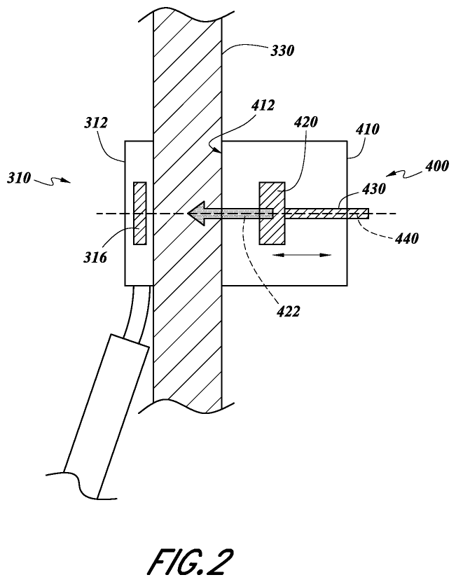

FIG. 2 schematically illustrates a cross-sectional view of an example apparatus 400 in accordance with certain implementations described herein. The example apparatus 400 can be configured to be positioned outside a recipient's body at a location at which an external portion of a transcutaneous system (e.g., external component 320) can be positioned, instead of the apparatus 400. For example, the apparatus 400 of FIG. 2 can be positioned at a location (e.g., on the recipient's tissue 330 overlaying an implantable component 310 of the transcutaneous auditory prosthesis system 300 implanted within the recipient's body) at which the external component 320 of the transcutaneous auditory prosthesis system 300 can be positioned, instead of the apparatus 400, such that the external component 320 is in wireless communication with the implantable component 310 and is retained at the location by a magnetic attraction between the implantable component 310 and the external component 320.

In certain implementations, the apparatus 400 comprises a housing 410 having an external surface 412 configured to be placed on a non-magnetic material (e.g., tissue 330) overlaying a first device (e.g., implantable component 310; medical implant) comprising a first ferromagnetic or ferrimagnetic material (e.g., at least one internal magnetic material 316). The apparatus 400 further comprises a second ferromagnetic or ferrimagnetic material 420 (e.g., at least one magnet) within the housing 410, the second ferromagnetic or ferrimagnetic material 420 configured to generate an attractive magnetic force 422 with the first device. For example, at least one of the first ferromagnetic or ferrimagnetic material and the second ferromagnetic or ferrimagnetic material 420 can comprise a magnetized material (e.g., magnet). The apparatus 400 further comprises a movable coupler 430 in mechanical communication with the housing 410 and the second ferromagnetic or ferrimagnetic material 420, the coupler 430 configured to controllably adjust a distance between the second ferromagnetic or ferrimagnetic material 420 and the first device along a longitudinal axis 440 substantially perpendicular to the external surface 412. While FIG. 2 schematically illustrates the coupler 430 being configured to be controllably and linearly slid along the longitudinal axis 440 to move the second ferromagnetic or ferrimagnetic material 420 relative to the external surface 412, other motions and/or mechanisms of the coupler 430 are also compatible with certain implementations described herein (e.g., the coupler 430 rotated along a screw thread encircling the longitudinal axis 440; different sized spacers inserted/removed from between the second ferromagnetic or ferrimagnetic material 420 and the external surface 412; different receptacles at different distances from the external surface 412, the coupler 430 configured to be inserted into a selected receptacle) to move the second ferromagnetic or ferrimagnetic material 420 relative to the external surface 412.

In certain implementations, the apparatus 400 is configured to approximate a mass, size, and/or shape of an external portion of a transcutaneous system (e.g., external component 320 of a transcutaneous auditory prosthesis system 300) and is configured to provide information regarding a thickness of the non-magnetic material overlaying the first device (e.g., SFT of the recipient's tissue 330), information regarding a magnitude of the attractive magnetic force 422, and/or information regarding a magnetic attraction strength sufficient to retain the external portion (e.g., external component 320) at the location. For example, the attractive magnetic force 422 can be configured to approximate a magnetic force between the implantable component 310 and an external component 320 to be placed at the location. If the external portion of the transcutaneous system comprises at least one magnet having multiple configurations, each configuration resulting in a corresponding magnetic force with the first device, then the attractive magnetic force 422 between the apparatus 400 and the first device can be configured to approximate the corresponding magnetic forces for one or more (e.g., each) of the multiple configurations. For another example, the apparatus 400 can be smaller and/or lighter than the external portion of the transcutaneous system, and the attractive magnetic forces 422 with the second ferromagnetic or ferrimagnetic material 420 of the apparatus 400 in the various positions and/or orientations can be weaker than, but correspond to, the magnetic forces of the various configurations of the external portion of the transcutaneous system.

In certain implementations, the housing 410 is configured to be placed on the recipient's body (e.g., on or over the recipient's skin). The housing 410 of certain implementations comprises at least one biocompatible material that is substantially transparent to the magnetic flux from the second ferromagnetic or ferrimagnetic material 420 such that the housing 410 does not substantially interfere with the magnetic attraction between the apparatus 400 and the first device. For example, the material of the housing 410 can comprise plastic (e.g., PEEK), silicone, or ceramic (e.g., zirconium oxide). The housing 410 can have a width (e.g., along a lateral direction substantially parallel to the recipient's skin) less than or equal to 40 millimeters (e.g., in a range of 15 millimeters to 35 millimeters; in a range of 25 millimeters to 35 millimeters; in a range of less than 30 millimeters; in a range of 15 millimeters to 30 millimeters).

In certain implementations in which the non-magnetic material comprises a recipient's tissue 330 (e.g., skin), the external surface 412 is configured to provide a sliding friction force with the recipient's tissue 330 that is substantially not dependent on a condition of the recipient's tissue. For example, the external surface 412 can comprise a plastic material configured to have a substantially consistent sliding friction force with the recipient's skin regardless of whether the recipient's skin is dry, oily, clammy, sweaty, etc. For another example, the housing 410 can comprise two facing surfaces contacting one another, substantially parallel to the external surface 412, and positioned between the external surface 412 and the second ferromagnetic or ferrimagnetic material 420. The two facing surfaces can have a predetermined sliding friction force therebetween, which can be helpful to provide consistency among various recipients when using the apparatus 400 to determine the recipient's actual SFT, as described more fully herein.

In certain implementations, at least one of the first ferromagnetic or ferrimagnetic material and the second ferromagnetic or ferrimagnetic material 420 comprises a magnet (e.g., a permanent magnet; an electromagnet) such that there is a magnetic attractive force between the first ferromagnetic or ferrimagnetic material and the second ferromagnetic or ferrimagnetic material 420. In certain implementations, the first device has a first dipole magnetic moment (e.g., the at least one internal magnetic material 316 of the implanted component 310 comprises at least one permanent magnet) and the second ferromagnetic or ferrimagnetic material 420 has a second dipole magnetic moment configured to interact with the first dipole magnetic moment to generate the attractive magnetic force 422.

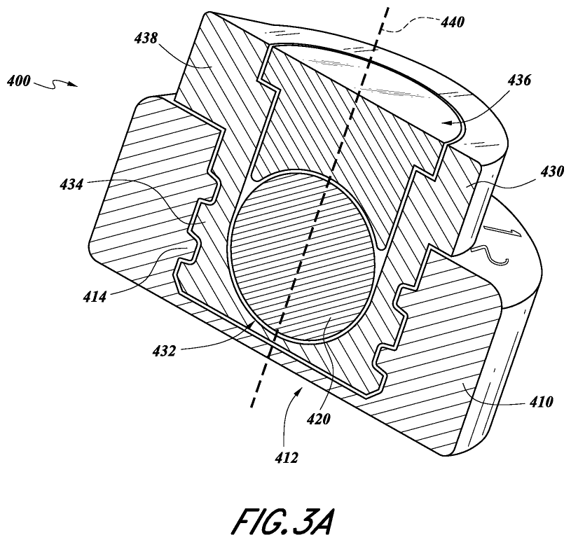

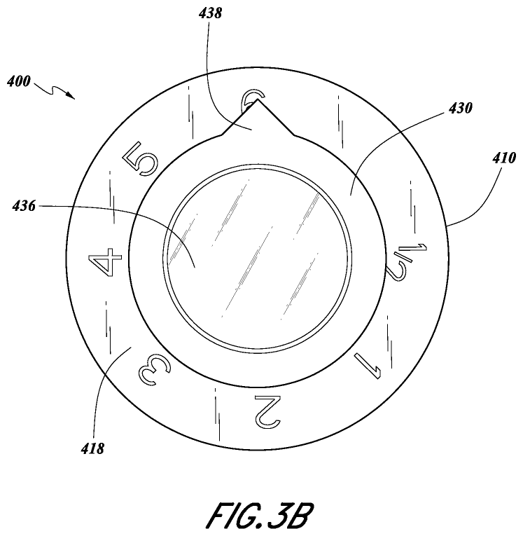

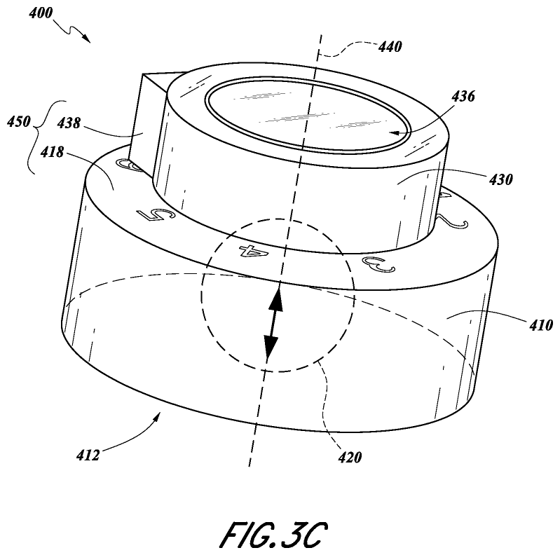

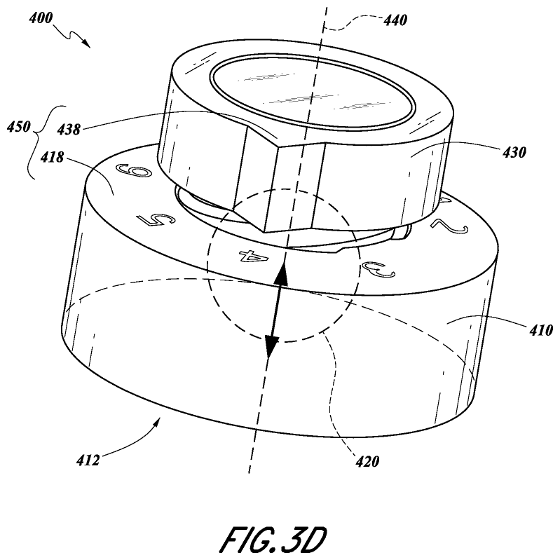

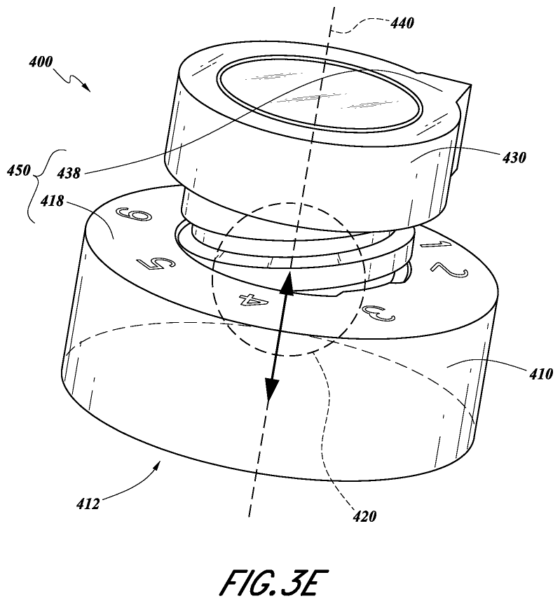

FIGS. 3A and 3B schematically illustrate a cross-sectional view and a top view, respectively, of an example apparatus 400 having an example movable coupler 430 (e.g., dial) in accordance with certain implementations described herein. FIGS. 3C-3E schematically illustrate perspective views of the example apparatus 400 of FIGS. 3A-3B in three different configurations with the coupler 430 at various positions and/or orientations relative to the housing 410 in accordance with certain implementations described herein. The example apparatus 400 of FIGS. 3A-3E is configured to adjust a strength of the attractive magnetic force 422 between the second ferromagnetic or ferrimagnetic material 420 and the first device. The coupler 430 of FIG. 3A comprises a receptacle 432 configured to hold the second ferromagnetic or ferrimagnetic material 420 and a screw thread 434 spiraled around the longitudinal axis 440. The screw thread 434 is configured to mate with a corresponding screw thread 414 of the housing 410, and the coupler 430 is configured to controllably move the second ferromagnetic or ferrimagnetic material 420 along the direction (e.g., along the longitudinal axis 440) by rotating about the longitudinal axis 440.

In certain implementations, the second ferromagnetic or ferrimagnetic material 420 is configured to freely rotate relative to the coupler 430. By freely rotating, the second ferromagnetic or ferrimagnetic material 420 of certain implementations is compatible for use with various types of first devices (e.g., first devices having axially magnetized magnets; first devices having diametrically magnetized magnets). For example, as shown in FIG. 3A, the second ferromagnetic or ferrimagnetic material 420 can have a substantially spherical shape and can be held within a substantially spherical receptacle 432 of the coupler 430 such that the second ferromagnetic or ferrimagnetic material 420 is free to rotate within the substantially spherical receptacle 432 (e.g., in response to the attractive magnetic force 422 and/or in response to rotation of the coupler 430). In certain other implementations, the second ferromagnetic or ferrimagnetic material 420 and/or the receptacle 432 have other shapes and/or the second ferromagnetic or ferrimagnetic material 420 and the coupler 430 are held together in other ways.

In certain implementations, the first device has a first dipole magnetic moment (e.g., a dipole magnetic moment of at least one permanent magnet of the at least one internal magnetic material 316), the second ferromagnetic or ferrimagnetic material 420 has a second magnetic moment, and the second ferromagnetic or ferrimagnetic material 420 is configured to rotate within the housing 410 such that the second dipole magnetic moment is aligned substantially parallel to the first dipole magnetic moment. For example, for a first device having a first dipole magnetic moment substantially parallel to the non-magnetic material (e.g., tissue 330) between the first device and the external surface 412 of the apparatus 400, in response to the attractive magnetic force 422 between the second ferromagnetic or ferrimagnetic material 420 and the first device, the second ferromagnetic or ferrimagnetic material 420 rotates such that the second dipole magnetic moment of the second ferromagnetic or ferrimagnetic material 420 is substantially parallel and opposite to the first dipole magnetic moment (e.g., substantially parallel to the non-magnetic material; substantially parallel to the external surface 412). For another example, for a first device having a first dipole magnetic moment substantially perpendicular to the non-magnetic material (e.g., tissue 330) between the first device and the external surface 412 of the apparatus 400, in response to the attractive magnetic force 422 between the second ferromagnetic or ferrimagnetic material 420 and the first device, the second ferromagnetic or ferrimagnetic material 420 rotates such that the second dipole magnetic moment of the second ferromagnetic or ferrimagnetic material 420 is substantially parallel and opposite to the first dipole magnetic moment (e.g., substantially perpendicular to the non-magnetic material; substantially perpendicular to the external surface 412).

In certain implementations, the second ferromagnetic or ferrimagnetic material 420 comprises a visual indication of an orientation of the dipole magnetic moment of the second ferromagnetic or ferrimagnetic material 420 and the apparatus 400 further comprises an optically transparent portion (e.g., window; opening) configured to allow a user to view the visual indication. For example, the second ferromagnetic or ferrimagnetic material 420 can have at least one color pattern and/or character that is indicative of the direction of the second dipole magnetic moment of the second ferromagnetic or ferrimagnetic material 420 and the coupler 430 can comprise a substantially optically transparent portion 436 (e.g., window; opening) through which the at least one color pattern and/or character is viewable by the user.

In the first configuration of FIG. 3C, the coupler 430 is positioned and/or orientated such that the second ferromagnetic or ferrimagnetic material 420 is at a first position relative to the external surface 412. In the second configuration of FIG. 3D, the coupler 430 is positioned and/or oriented such that the second ferromagnetic or ferrimagnetic material 420 is at a second position relative to the external surface 412, the second position farther from the external surface 412 than is the first position. In the third configuration of FIG. 3E, the coupler 430 is positioned and/or orientated such that the second ferromagnetic or ferrimagnetic material 420 is at a third position relative to the external surface 412, the third position farther from the external surface 412 than is the second position. For the example apparatus 400 of FIGS. 3C-3E, the second ferromagnetic or ferrimagnetic material 420 can be moved among the first, second, and third positions relative to the external surface 412 by rotating (e.g., dialing) the coupler 430 such that the screw threads 434 of the coupler 430 travel along the screw threads 414 of the housing 410 (e.g., screwing and unscrewing the coupler 430 into and out of the housing 410). In this way, the coupler 430 (e.g., dial) can be rotated to adjust a strength of the attractive magnetic force 422 between the second ferromagnetic or ferrimagnetic material 420 and the first device. In certain implementations, the coupler 430 comprises a ratchet mechanism (e.g., ratchet stop) to facilitate consistent and reliable (e.g., repeatable) measurements.

With the apparatus 400 at the location on the non-magnetic material overlaying the first device, the first position of the second ferromagnetic or ferrimagnetic material 420 corresponds to a first distance between the second ferromagnetic or ferrimagnetic material 420 and the first device, the second position of the second ferromagnetic or ferrimagnetic material 420 corresponds to a second distance between the second ferromagnetic or ferrimagnetic material 420 and the first device (the second distance larger than the first distance), and the third position of the second ferromagnetic or ferrimagnetic material 420 corresponds to a third distance between the second ferromagnetic or ferrimagnetic material 420 and the first device (the third distance larger than the second distance). By being at the location on the non-magnetic material overlaying the first device, the various distances between the second ferromagnetic or ferrimagnetic material 420 and the first device correspond to various strengths of the attractive magnetic force 422 between the second ferromagnetic or ferrimagnetic material 420 and the first device (e.g., the strength of the attractive magnetic force 422 in the first configuration of FIG. 3C stronger than the strength of the attractive magnetic force 422 in the second configuration, which is stronger than the strength of the attractive magnetic force 422 in the third configuration). With the apparatus 400 retained on the non-magnetic material and in an example configuration in which the coupler 430 is at an example location and/or orientation relative to the housing 410 (e.g., the second ferromagnetic or ferrimagnetic material 420 at an example position relative to the external surface 412; the second ferromagnetic or ferrimagnetic material 420 at an example distance from the first device resulting in an example attractive magnetic force 422 with the first device), a user of the apparatus 400 can determine whether the example attractive magnetic force 422 for the example configuration of the apparatus 400 is sufficient to retain the apparatus 400 on the non-magnetic material (e.g., by observing whether the apparatus 400 detaches from the non-magnetic material while the apparatus 400 is in the example configuration).

For example, the apparatus 400 can be on the recipient's tissue 330 in place of the external component 320 of a transcutaneous system 300 (e.g., a transcutaneous auditory prosthesis system) and in an example configuration in which the second ferromagnetic or ferrimagnetic material 420 is at an example distance from the implantable component 310 of the transcutaneous system 300, and a user of the apparatus 400 can observe whether the apparatus 400 remains retained on the recipient's tissue 330 by the example attractive magnetic force 422. If the apparatus 400 becomes detached from the recipient's tissue 330 while being exposed to expected operating conditions (e.g., a range of forces and/or torques applied to the apparatus 400 and/or the recipient due to movements or other behaviors and/or experiences by the recipient), then the example attractive magnetic force 422 can be deemed to be insufficient to retain the apparatus 400 on the recipient's tissue 330. In certain implementations in which the apparatus 400 is configured to emulate (e.g., simulate) an external component 320 of a transcutaneous system 300 (e.g., to approximate a mass, size, and/or shape of the external component 320) with the attractive magnetic force 422 emulating (e.g., simulating; approximating) the magnetic attraction of the external component 320 with the implanted component 310, a determination of the insufficiency of the attractive magnetic force 422 to retain the apparatus 400 on the recipient's tissue 330 can be considered by the user while selecting an appropriate external component 320 having an appropriate magnetic attraction with the implanted component 310 (e.g., sufficient to retain the external component 320 on the recipient's tissue 330 while being exposed to the expected operating conditions for the external component 320) to be used in the transcutaneous system 300.

In certain implementations, the coupler 430 comprises at least one indicator 450 of a position and/or orientation of the coupler 430 relative to the housing 410. As shown in FIGS. 3A-3E, the coupler 430 can comprise a first asymmetric portion 438 (e.g., at least one protrusion, recess, color pattern, series of markings or gradations, and/or alphanumeric character) and the housing 410 can comprise a second asymmetric portion 418 (e.g., at least one protrusion, recess, color pattern, series of markings or gradations, and/or alphanumeric character) such that the relative position and/or orientation of the first asymmetric portion 438 relative to the second asymmetric portion 418 is indicative of the position and/or orientation of the coupler 430 relative to the housing 410. For example, the first asymmetric portion 438 can comprise a protrusion (e.g., pointer) of the coupler 430 and the second asymmetric portion 418 can comprise a plurality of indicia (e.g., series of markings or gradations; alphanumeric characters) on a viewable surface of the housing 410. The protrusion can extend radially relative to the longitudinal axis 440, and the plurality of indicia can be positioned around the longitudinal axis 440. Other types of the at least one indicator 438 are also compatible with certain implementations described herein. In addition, the at least one indicator 438 of certain other implementations can comprise at least one sensor (e.g., optical sensor; electrical sensor) configured to generate a sensor signal indicative of the position and/or orientation of the coupler 430 relative to the housing 410, at least one signal processor (e.g., microcontroller) configured to generate a display control signal in response to the sensor signal, and at least one display device (e.g., LED lights; alphanumeric display device) configured to display at least one color pattern and/or alphanumeric character indicative of the position and/or orientation of the coupler 430 relative to the housing 410. In certain other implementations, instead of comprising at least one display device and at least one signal processor, the apparatus 400 is configured to wirelessly transmit the sensor signal to another device comprising at least one display device.

In certain implementations, the at least one indicator 450 is configured to provide information regarding the retention magnets that can be selected for the second device, the appropriate retention magnet to select for the second device, the attractive magnetic force 422 generated by the apparatus 400, and/or the thickness of the non-magnetic material between the first device and the external surface 412 of the apparatus 400. The indicia of the at least one indicator 450 can comprise a scale which is configured (e.g., calibrated) to convert the position and/or orientation of the second ferromagnetic or ferrimagnetic material 420 into the information to be provided to the user (e.g., alphanumeric characters corresponding to the different retention magnets available for the second device). For an apparatus 400 configured to provide information regarding multiple types of first devices and/or second devices, the at least one indicator 450 can comprise multiple scales, each scale configured (e.g., calibrated) to convert the position and/or orientation of the second ferromagnetic or ferrimagnetic material 420 into the information relevant to a corresponding first device and/or second device. For example, an apparatus 400 configured to emulate both axially magnetized retention magnets and diametrically magnetized retention magnets, the at least one indicator 450 can comprise two scales each calibrated to correspond to a respective retention magnet type.

For example, to provide information for determining whether a particular recipient with a particular implantable component 310 of a transcutaneous auditory prosthesis system 300 is able to use at least one of the different retention magnet configurations (e.g., retention magnet strengths) that are available for an external component 320 and/or to provide information for determining which of the different retention magnet configurations is appropriate to use with the particular recipient, the indicia of at least one indicator 450 can be scaled (e.g., calibrated) to convert the position and/or orientation of the second ferromagnetic or ferrimagnetic material 420 into the different retention magnet configurations of the external component 320 being emulated by the different positions and/or orientations of the second ferromagnetic or ferrimagnetic material 420. With the apparatus 400 configured to emulate a particular retention magnet configuration, if the apparatus 400 is not retained by the attractive magnetic force 422 (e.g., becomes detached from the recipient's tissue 330 while being exposed to expected operating conditions), then the particular retention magnet configuration can be deemed to be inappropriate for use by the particular recipient. Alternatively, if the apparatus 400 is retained by the attractive magnetic force 422, then the particular retention magnet configuration can be deemed to be appropriate for use by the particular recipient. If none of the retention magnet configurations available for the external component 320 are deemed appropriate for use by the particular recipient, then the external component 320 can be deemed to be inappropriate for use by the particular recipient, and an alternative external component 320 having alternative retention magnet configurations may be appropriate.

For another example, to provide information for determining the actual SFT of the recipient's tissue 330 between the implantable component 310 and the external surface 412 of the apparatus 400, the indicia of at least one indicator 450 can be scaled (e.g., calibrated) to convert the position and/or orientation of the second ferromagnetic or ferrimagnetic material 420 into different hypothetical SFT values for the recipient's tissue 330 overlaying a particular type of implantable component 310. With the apparatus 400 having an attractive magnetic force 422 that is expected to retain the apparatus 400 on the recipient's tissue 330 with a particular hypothetical SFT value, if the apparatus 400 is not retained by the attractive magnetic force 422 (e.g., becomes detached from the recipient's tissue 330 while being exposed to expected operating conditions), then the actual SFT value of the recipient's tissue 330 can be deemed to be larger than the particular hypothetical SFT value. Alternatively, if the apparatus 300 is retained by the attractive magnetic force 422, then the actual SFT value of the recipient's tissue 330 can be deemed to be equal to or less than the particular hypothetical SFT value. Multiple iterations of using the apparatus 400 in this way, each iteration having a different hypothetical SFT value, can be used to evaluate the actual SFT value of the recipient's tissue 330.

For an apparatus 400 configured to provide information regarding appropriate retention magnet configurations and actual SFT values, the at least one indicator 450 can comprise multiple scales, at least one scale configured (e.g., calibrated) to convert the position and/or orientation of the second ferromagnetic or ferrimagnetic material 420 into the information relevant to appropriate retention magnet configurations and at least one scale configured (e.g., calibrated) to convert the position and/or orientation of the second ferromagnetic or ferrimagnetic material 420 into the hypothetical SFT values. Alternatively, the at least one indicator 450 can comprise a single scale, and readings from the scale can be converted to the corresponding values using a conversion table, conversion algorithm, computer software, etc. so that the scale reading provides the desired information (e.g., magnet strength; estimate of the actual SFT value).

In certain implementations, the coupler 430 is continually adjustable (e.g., configured to position and/or orient the second ferromagnetic or ferrimagnetic material 420 at a continuous series of positions and/or orientations) relative to the housing 410. The housing 410 and the coupler 430 can have sufficient friction therebetween to hold the coupler 430 in place, or the coupler 430 and/or the housing 410 can comprise a locking mechanism (e.g., actuator configured to engage or otherwise hold the coupler 430 in place). In certain other implementations, the coupler 430 is configured to have a finite set of discrete positions and/or orientations relative to the housing 410. The finite set of discrete positions and/or orientations can correspond to attractive magnetic forces 422 that correspond to (e.g., substantially equal to; approximate) a finite set of magnetic attractions generated by a second device having a finite set of magnetic configurations upon the second device being placed at the location on the non-magnetic material overlaying the first device. In certain such implementations, the second device can comprise at least one retention magnet (e.g., the external magnetic material 326 of the external component 320) that can be placed in a plurality of magnetic configurations (e.g., positions and/or orientations of the at least one retention magnet), each magnetic configuration having a different magnetic attraction to the first device upon the second device being placed at the location on the non-magnetic material overlaying the first device. In certain other such implementations, the second device can be compatible to receive at least one retention magnet selected from a plurality of different retention magnets having different magnetic attractions to the first device upon being received by the second device and the second device being placed at the location on the non-magnetic material overlaying the first device. For example, an external component 320 (e.g., external transmitter unit 128) of a transcutaneous auditory prosthesis system 300 can be configured to receive a retention magnet selected from a finite set (e.g., two, three, four, five, six, or more) of types of retention magnets, each type of retention magnet having a different magnetic flux magnitude. Each of the discrete positions and/or orientations of the coupler 430 can have the second ferromagnetic or ferrimagnetic material 420 positioned and/or oriented to produce a corresponding attractive magnetic force 422 that approximates (e.g., is substantially equal to) a magnetic attraction resulting from the selected retention magnet being used in the external component 320.

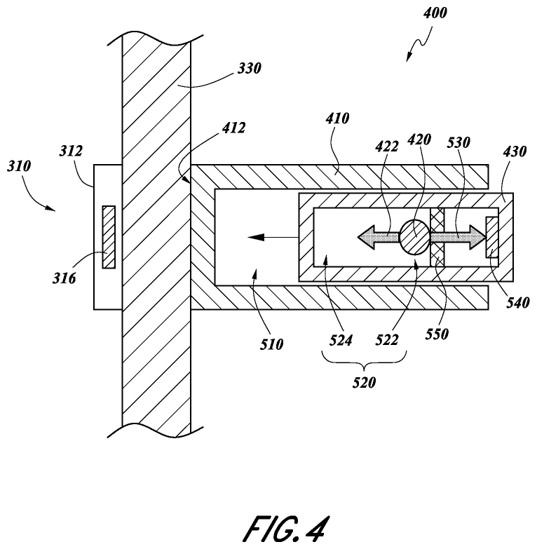

FIG. 4 schematically illustrates a cross-sectional view of an example apparatus 400 having a coupler 430 configured to be controllably and linearly slid along the longitudinal axis 440 relative to the housing 410 in accordance with certain implementations described herein. In certain implementations, the distance between the second ferromagnetic or ferrimagnetic material 420 and the first device along the longitudinal axis 440 substantially perpendicular to the external surface 412 can be controllably adjusted by controllably sliding the coupler 430 relative to the housing 410. The coupler 430 can comprise one or more features described above with regard to the example coupler 430 of FIGS. 3A-3E (e.g., configured to allow the second ferromagnetic or ferrimagnetic material 420 to freely rotate; a substantially optically transparent portion 436 through which the orientation of the second ferromagnetic or ferrimagnetic material 420 is viewable by the user; at least one indicator 450 of a position and/or orientation of the coupler 430 relative to the housing 410; continually adjustable at a continuous series of positions and/or orientations relative to the housing 410; having a finite set of discrete positions and/or orientations relative to the housing 410).

In certain implementations, the housing 410 comprises a cylindrically shaped region 510 in which the coupler 430 can be received and controllably slid. For example, as shown in FIG. 4, the housing 410 can comprise a first tube having a closed end comprising the external surface 412 and the coupler 430 can comprise a second tube within the first tube, the second tube having two closed ends. Other shapes and configurations of the housing 410 and the coupler 430 are also compatible with certain implementations described herein.

In certain implementations, the coupler 430 comprises a cavity 520 containing the second ferromagnetic or ferrimagnetic material 420, the cavity 520 can comprise a first region 522 in which the second ferromagnetic or ferrimagnetic material 420 is held by a retention force 530 and a second region 524 into which the second ferromagnetic or ferrimagnetic material 420 is configured to move in response to the attractive magnetic force 422 being greater than the retention force 530. In certain implementations, the direction of the retention force 530 is substantially opposite to the direction of the attractive magnetic force 422 applied to the at least one magnet 420 by the first device and is substantially unchanging in both magnitude and direction as the coupler 430 is controllably slid along the housing 410. As shown in FIG. 4, the retention force 530 of certain implementations is a second attractive magnetic force on the second ferromagnetic or ferrimagnetic material 420 generated by at least one fixed magnet 540. The retention force 530 of certain other implementations comprises a spring force (e.g., generated by a mechanical spring or an air spring in mechanical communication with the second ferromagnetic or ferrimagnetic material 420), an adhesive force (e.g., generated by an adhesive material between the at least one magnet 420 and an internal surface of the coupler 430), a weak mechanical bonding force (e.g., generated by a hook-and-loop fastener such as a Velcro® fastener between the at least one magnet 420 and an internal surface of the coupler 430), and/or another type of force generated by another type of structure.

FIG. 4 shows an example in which the retention force 530 is a second attractive magnetic force. The coupler 430 of FIG. 4 further comprises at least one fixed magnet 540 and a partition 550 comprising a non-magnetic material and at least partially bounding the first region 522 and/or the cavity 520. The partition 550 is between the second ferromagnetic or ferrimagnetic material 420 and the at least one fixed magnet 540 (e.g., the at least one fixed magnet 540 is outside the cavity 520). The at least one fixed magnet 540 is in magnetic communication with the second ferromagnetic or ferrimagnetic material 420 to generate the retention force 530 on the second ferromagnetic or ferrimagnetic material 420. In certain implementations, the at least one fixed magnet 540 comprises a permanent magnet that is substantially identical to a permanent magnet (e.g., the at least one internal magnetic material 316) of the first device.

In an example use of the apparatus 400 of FIG. 4 in conjunction with an implantable component 310 beneath the recipient's tissue 330, the apparatus 400 can be placed on the recipient's tissue 330 with the second ferromagnetic or ferrimagnetic material 420 in the first region 522 of the cavity 520 and the coupler 430 at a position sufficiently far from the external surface 412 such that the retention force 530 on the second ferromagnetic or ferrimagnetic material 420 is greater than the attractive magnetic force 422 applied to the second ferromagnetic or ferrimagnetic material 420 by the magnetic interaction of the second ferromagnetic or ferrimagnetic material 420 with the at least one internal magnetic material 316. Initially, as the coupler 430 is controllably and linearly slid along the longitudinal axis 440 towards the external surface 412, the second ferromagnetic or ferrimagnetic material 420 remains in the first region 522 and is moved closer to the implantable component 310, with the attractive magnetic force 422 becoming larger due to the reduction of the distance between the second ferromagnetic or ferrimagnetic material 420 and the implantable component 310. At a position at which the attractive magnetic force 422 becomes larger than the retention force 530, the second ferromagnetic or ferrimagnetic material 420 moves from the first region 522 to the second region 524 of the cavity 520, thereby signaling that the attractive magnetic force 422 is greater than the retention force 530. The second ferromagnetic or ferrimagnetic material 420 of certain implementations, is configured to return to the first region 522 upon the attractive magnetic force 422 being less than the retention force 530.

In certain implementations, the movement of the second ferromagnetic or ferrimagnetic material 420 from the first region 522 to the second region 524 causes an audible sound (e.g., click) due to the second ferromagnetic or ferrimagnetic material 420 hitting the end portion of the coupler 430 closest to the implantable component 310. For example, the second region can comprise a metallic plate or other component configured to increase the sound of the second ferromagnetic or ferrimagnetic material 420 hitting the end portion of the coupler 430. In certain other implementations, the housing 410 and/or the coupler 430 comprises a substantially optically transparent portion (e.g., window; opening) configured to allow a user to visually see the position of the second ferromagnetic or ferrimagnetic material 420 (e.g., whether the second ferromagnetic or ferrimagnetic material 420 is in the first region 522 or the second region 524). In certain other implementations, the second ferromagnetic or ferrimagnetic material 420 is mechanically coupled to an indicator (e.g., plastic piece having two adjacent differently colored regions) visible through a substantially optically transparent portion of the housing 410 and/or the coupler 430 such that a first portion of the indicator (e.g., first colored region) is visible when the second ferromagnetic or ferrimagnetic material 420 is in the first region 522 and a second portion of the indicator (e.g., second colored region) is visible when the second ferromagnetic or ferrimagnetic material 420 is in the second region 524. In still other implementations, the apparatus 400 comprises at least one sensor (e.g., optical sensor; infrared sensor; electrical sensor; magnetic sensor; mechanical switch) configured to generate a sensor signal indicative of the position of the second ferromagnetic or ferrimagnetic material 420 relative to the coupler 430 (e.g., the at least one sensor comprising the second ferromagnetic or ferrimagnetic material 420), at least one signal processor (e.g., microcontroller) configured to generate a display control signal in response to the sensor signal, and at least one indicator (e.g., LED light; alphanumeric display device; sound speaker) configured to present a user-receivable signal (e.g., light; pattern; alphanumeric character; sound) indicative of the position of the second ferromagnetic or ferrimagnetic material 420 relative to the coupler 430 (e.g., to indicate that the second ferromagnetic or ferrimagnetic material 420 has moved to/from the first region 522 and/or the second region 524). In certain other implementations, instead of comprising at least one display device and at least one signal processor, the apparatus 400 is configured to wirelessly transmit the sensor signal to another device comprising at least one display device.

In certain implementations, the housing 410 and/or the coupler 430 comprises indicia (e.g., a series of markings, gradations, and/or alphanumeric characters) configured to provide information regarding the position of the coupler 430 relative to the housing 410 (e.g., using a predetermined relationship between the magnetic strength of the at least one internal magnetic material 316 and the strength of the retention force 530 on the second ferromagnetic or ferrimagnetic material 420 in the first region 522 by the at least one fixed magnet 540). For example, the indicia can be scaled to equate positions of the coupler 430 at which the second ferromagnetic or ferrimagnetic material 420 moves from the first region 522 to the second region 524 to actual SFT values of the recipient's tissue 330. Such an apparatus 400 can provide a measurement indicative of the actual SFT value of the recipient's tissue 330. For another example, the indicia can be scaled to equate positions of the coupler 430 at which the second ferromagnetic or ferrimagnetic material 420 moves from the first region 522 to the second region 524 to a finite set of magnetic attractions generated by an external component 320 in a finite set of magnetic configurations upon the external component 320 being placed at the location on the recipient's tissue 330 overlaying the implantable component 310. Such an apparatus 400 can provide a measurement indicative of an acceptable magnetic configuration for the external component 320 to be used by the recipient.

In certain implementations, the pressure applied by the apparatus 400 on the recipient's tissue 330 is dependent on the orientation of the apparatus 400 relative to the recipient's tissue 330 and to the ground. In addition, the strength of the retention force 530 that overcomes the strength of the magnetic attractive force 422 is dependent on the orientation of the apparatus 400 relative to the ground (e.g., whether the retention force 530 also has to overcome the weight of the second ferromagnetic or ferrimagnetic material 420). As such, whether the apparatus 400 is calibrated to denote pressure applied to the recipient's tissue 330 and/or thickness of the recipient's tissue 330 (e.g., skin flap thickness) depends on the orientation of the apparatus 400 relative to the recipient's tissue 330 and to the ground. In certain such implementations, the apparatus 400 is configured to provide calibrated values of pressure and/or tissue thickness upon the apparatus 400 being operated with the recipient's tissue 330 and the apparatus 400 both in predetermined orientations relative to the ground. The apparatus 400 can comprise a sensor (e.g., spirit level; bubble level; accelerometer) configured to detect the orientation of the apparatus 400 relative to the ground (e.g., the Earth) to assist a user to orient the apparatus 400 consistently and/or in accordance with the predetermined orientation.

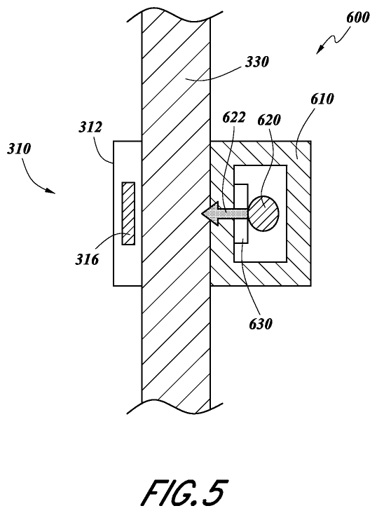

FIG. 5 schematically illustrates a cross-sectional view of an example apparatus 600 comprising a gauge 630 in accordance with certain implementations described herein. The apparatus 600 comprises a housing 610 (e.g., comprising at least one biocompatible material that is substantially transparent to the magnetic flux from the second ferromagnetic or ferrimagnetic material 620) configured to be placed over a non-magnetic material (e.g., recipient's tissue 330) overlaying a device (e.g., implantable component 310) comprising a first ferromagnetic or ferrimagnetic material (e.g., at least one internal magnetic material 316). The apparatus 600 further comprises a second ferromagnetic or ferrimagnetic material 620 within the housing 610, the second ferromagnetic or ferrimagnetic material 620 configured to generate an attractive magnetic force 622 with the device. The apparatus 600 further comprises a gauge 630 within the housing 610 and in mechanical communication with the second ferromagnetic or ferrimagnetic material 620. The gauge 630 is configured to generate an indication of a magnitude of the attractive magnetic force 622 along a direction towards the device and/or a thickness of the non-magnetic material between the device and the housing 610.

In certain implementations, the second ferromagnetic or ferrimagnetic material 620 comprises a permanent magnet, while in certain other implementations, the second ferromagnetic or ferrimagnetic material 620 is configured to be attracted to a magnetic force generated by the at least one internal magnetic material 316. In certain implementations, the second ferromagnetic or ferrimagnetic material 620 comprises a core of an electromagnet which is configured to generate a magnetic field that interacts with the at least one internal magnetic material 316 to produce the attractive magnetic force 622. In certain other implementations, in place of the second ferromagnetic or ferrimagnetic material 620 of FIG. 5, the apparatus 600 comprises an electromagnet without a core.

While FIG. 5 shows the gauge 630 located directly between the at least one internal magnetic material 316 and the second ferromagnetic or ferrimagnetic material 620 and configured to measure a compression force, the gauge 630 can be located to one side of the second ferromagnetic or ferrimagnetic material 620 (e.g., attached to the housing 610 and to the second ferromagnetic or ferrimagnetic material 620) and configured to measure a tension force. In certain implementations, the indication of the magnitude of the attractive magnetic force 622 is expressed as a magnitude of a force sufficient to remove the apparatus 600 from the location on the non-magnetic material overlaying the device.

In certain implementations, the apparatus 600 comprising the gauge 630 does not have the functionality of an external component 320 configured to operate in conjunction with the implanted component 310 during operation of the implanted component 310. For example, the implanted component 310 can comprise a cochlear implant configured to operate in conjunction with an external component 320 comprising a sound processor, and the apparatus 600 can be configured to be placed over the implanted component 310 (e.g., magnetically retained by the cochlear implant) and, while not having the functionality of the sound processor of the external component 320, can provide information (e.g., evaluate the actual SFT value of the recipient's tissue 330) useful for informing a selection of a magnetic configuration for an external component 320 comprising a sound processor to be used with the cochlear implant. In certain other implementations, the apparatus 600 comprising the gauge 630 does have the functionality of an external component 320 configured to operate in conjunction with the implanted component 310 during operation of the implanted component 310. For example, the implanted component 310 can comprise a cochlear implant configured to operate in conjunction with an external component 320 comprising a sound processor, and the apparatus 600 can be such an external component 320 configured to be placed over the implanted component 310 (e.g., magnetically retained by the cochlear implant), to operate in conjunction with the implanted component 310 to provide a hearing percept to the recipient, and to generate information (e.g., evaluate the actual SFT value of the recipient's tissue 330) useful for informing a selection of a magnetic configuration for the apparatus 600.

In certain implementations, the gauge 630 comprises a piezoelectric element between the second ferromagnetic or ferrimagnetic material 620 and the housing 610, the piezoelectric element configured to generate an electrical signal in response to the attractive magnetic force 622. The gauge 630 further comprises circuitry configured to respond to the electrical signal by generating the indication of the magnitude of the attractive magnetic force 622. For example, the circuitry can access a lookup table or a conversion algorithm configured to convert the measured attractive magnetic force 622 into a estimation of a thickness of the non-magnetic material overlaying the device (e.g., actual SFT of the recipient's tissue 330) and/or an estimation of a magnetic attraction strength sufficient to retain the external portion (e.g., external component 320) at the location. Other types of gauges 630 (e.g., mechanical spring; air spring) are also compatible with certain implementations described herein.

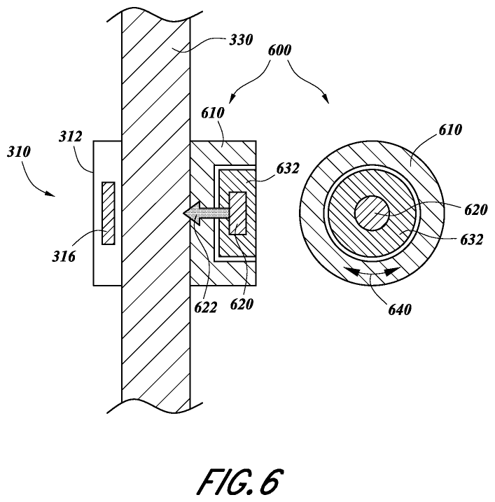

FIG. 6 schematically illustrates a side cross-sectional view and a top cross-sectional view of another example apparatus 600 comprising a gauge 630 in accordance with certain implementations described herein. The gauge 630 of FIG. 6 comprises a rotatable portion 632 (e.g., spinner) between the second ferromagnetic or ferrimagnetic material 620 and the housing 610. The rotatable portion 632 is configured to be controllably rotated relative to the housing 610 about the direction towards the device by a manually applied torque 640. The rotatable portion 632 is pressed against the housing 610 due to the attractive magnetic force 622 on the second ferromagnetic or ferrimagnetic material 620. To rotate the rotatable portion 632, the manually applied torque 640 is greater than a magnetically generated torque between the rotatable portion 632 and the housing 610, the magnetically generated torque resulting from a friction force between the rotatable portion 632 and the housing 610 due to the attractive magnetic force 622 between the second ferromagnetic or ferrimagnetic material 620 and the device. The indication of the magnitude of the attractive magnetic force 622 is the magnitude of the manually applied torque sufficient to rotate the rotatable portion 632 relative to the housing 610.

In certain other implementations, the gauge 630 comprises a laterally slidable portion (e.g., slider) between the second ferromagnetic or ferrimagnetic material 620 and the housing 610. The laterally slidable portion is configured to be controllably slid relative to the housing 610 in at least one direction substantially perpendicular to the direction towards the device by a manually applied force. The laterally slidable portion is pressed against the housing 610 due to the attractive magnetic force 622 on the second ferromagnetic or ferrimagnetic material 620. To slide the laterally slidable portion, the manually applied force is greater than a magnetically generated friction force between the laterally slidable portion and the housing 610, the magnetically generated friction force resulting from friction between the laterally slidable portion and the housing 610 due to the attractive magnetic force 622 between the second ferromagnetic or ferrimagnetic material 620 and the device. The indication of the magnitude of the attractive magnetic force 622 is the magnitude of the manually applied force sufficient to slide the laterally slidable portion relative to the housing 610.

In certain implementations, the second ferromagnetic or ferrimagnetic material 620 is affixed to the rotatable portion 632 or the laterally slidable portion, while in certain other implementations, the second ferromagnetic or ferrimagnetic material 620 and the rotatable portion 632 or the laterally slidable portion are configured to move relative to one another. In certain implementations in which the device comprises an implanted component 310 of a transcutaneous auditory prosthesis system 300, the attractive magnetic force 622 is configured to approximate a magnetic force between the implanted component 310 and an external component 320 (e.g., sound processor) of the transcutaneous auditory prosthesis system 300. In certain implementations, the apparatus 600 further comprises a material between and in contact with the housing 610 and the rotatable portion 632 or the laterally slidable portion, the material configured to provide a predetermined and substantially consistent friction force therebetween, which can be helpful to normalize the friction force created by the attractive magnetic force 622 and to provide consistency among various measurements using the gauge 630.

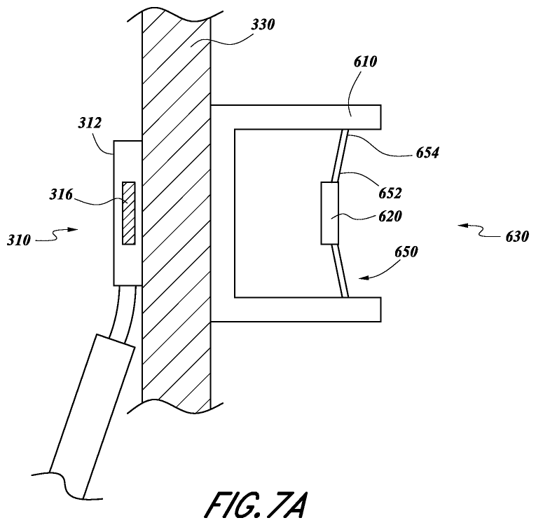

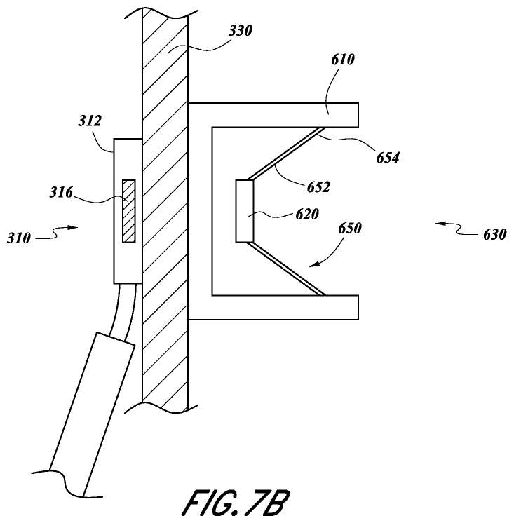

FIGS. 7A and 7B schematically illustrate two cross-sectional views of still another example apparatus 600 comprising a gauge 630 in accordance with certain implementations described herein. The gauge 630 of FIGS. 7A and 7B comprises at least one elastically deflectable element 650 (e.g., membrane; flexible arm) having a first portion 652 in mechanical communication with the second ferromagnetic or ferrimagnetic material 620 and a second portion 654 in mechanical communication with the housing 610. The at least one element 650 is configured to deflect under the attractive magnetic force 622 between the second ferromagnetic or ferrimagnetic material 620 and the implanted component 310. For example, for a relatively large thickness of the recipient's tissue 330 (e.g., a large SFT), the attractive magnetic force 622 deflects the at least one element 650 by a smaller amount (see, e.g., FIG. 7A) than for a relatively small thickness of the recipient's tissue 330 (e.g., a small SFT) (see, e.g., FIG. 7B).

In certain implementations, the indication of the magnitude of the attractive magnetic force 622 is the magnitude of the deflection of the at least one element 650. The housing 610 can comprise an optically transparent portion (e.g., window; opening) configured to allow a user to view the magnitude of the deflection. For example, the user can view the distance that the at least one element 650 and/or the second ferromagnetic or ferrimagnetic material 620 has moved by virtue of the deflection. For another example, the at least one element 650 comprises a material having visual properties (e.g., color; transparency) which depend on the amount of strain applied to the at least one element 650 or the viewing angle, and the visual properties can be viewable by the user through the optically transparent portion.

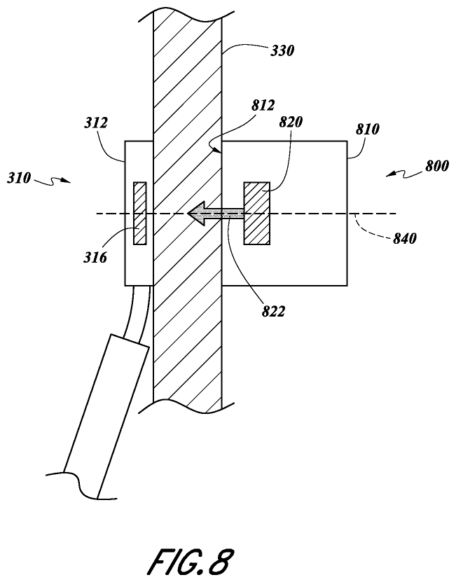

FIG. 8 schematically illustrates a cross-sectional view of an example apparatus 800 in accordance with certain implementations described herein. In certain implementations, the apparatus 800 comprises a housing 810 having an external surface 812 configured to be placed on a non-magnetic material (e.g., tissue 330) overlaying an implantable component 310 (e.g., medical implant) comprising a first ferromagnetic or ferrimagnetic material (e.g., at least one internal magnetic material 316). The apparatus 800 further comprises an electromagnet 820 within the housing 810, the electromagnet 820 configured to generate and controllably adjust an attractive magnetic force 822 between the electromagnet 820 and the implantable component 310 along a longitudinal axis 840 substantially perpendicular to the external surface 812.

In certain implementations, the electromagnet 820 comprises at least one electrically conductive conduit (e.g., wire coil) configured to generate a magnetic field in response to electrical current flowing through the at least one electrically conductive conduit. For example, the electromagnet 820 can comprise a magnetic core material around which the at least one electrically conductive conduit extends (e.g., a wire coil wrapped around a ferrite core). The apparatus 800 can further comprise control circuitry (e.g., integrated circuitry; microcontroller) comprising at least one portion (e.g., switch; variable resistor; variable transformer) in electrical communication with the at least one electrically conductive conduit and configured to receive electrical current from an electrical current source (e.g., battery of the apparatus 800; electrical conduit in electrical communication with an electric current source separate from the apparatus 800) and to controllably vary the electrical current flowing through the at least one electrically conductive conduit, thereby controllably varying the magnetic fields generated by the electromagnet 820. In this way, the control circuitry can controllably vary the attractive magnetic force 822 between the apparatus 800 and the implantable component 310. The attractive magnetic force 822 resulting from the electromagnet 820 can be controllably adjusted by the control circuitry to approximate the attractive magnetic force of a particular magnetic configuration of an external device to be placed in the location in place of the apparatus 800 (e.g., an external component 320 comprising a sound processor to be used with the implantable device 310).

The apparatus 800 can further comprise detection circuitry (e.g., sensor; gauge 630) configured to generate signals corresponding to the attractive magnetic force 822. For example, the apparatus 800 can comprise a sensor configured to generate signals indicative of the electrical current flowing through the electromagnet 820 (e.g., an ammeter portion of the control circuitry). For another example, the apparatus 800 can comprise a gauge (e.g., gauge 630) configured to generate signals indicative of a magnitude of the attractive magnetic force 822 along a direction towards the implantable component 310 (e.g., pressure against the tissue 330) and/or signals indicative of a thickness of the non-magnetic material (e.g., SFT of the recipient's tissue 330) between the implantable component 310 and the housing 810. The control circuitry can be further configured to receive the indications from the detection circuitry and to transmit signals to a display device of the apparatus 800 (e.g., LED lights; alphanumeric display device) and/or a display device (e.g., smartphone; smart tablet; computer) separate from the apparatus 800 to present information regarding the magnitude of the attractive magnetic force 822 and/or a thickness of the non-magnetic material to a user. For example, the display device can identify the particular magnetic configuration of the external device (e.g., external component 320) which is currently being approximated by the electromagnet 820 and/or the estimated SFT of the tissue 330.

In certain implementations in which the apparatus 800 does not have the functionality of an external component 320 configured to operate in conjunction with the implanted component 310 during operation of the implanted component 310, the apparatus 800 can be configured to be positioned outside a recipient's body at a location at which the external component 320 can be positioned (e.g., on the recipient's tissue 330 overlaying an implantable component 310 of the transcutaneous auditory prosthesis system 300 implanted within the recipient's body), instead of the apparatus 800. The apparatus 800 can be subsequently removed from the location and the external component 320 can be positioned at the location instead of the apparatus 800, such that the external component 320 is in wireless communication with the implantable component 310 and is retained at the location by a magnetic attraction between the implantable component 310 and the external component 320. In certain other implementations in which the apparatus 800 does have the functionality of an external component 320 configured to operate in conjunction with the implanted component 310 during operation of the implanted component 310, the apparatus 800 is configured to remain at the location (e.g., not removed from the location) and to be in wireless communication with the implantable component 310.

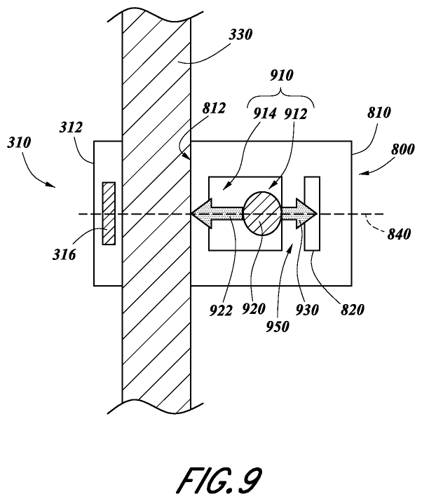

FIG. 9 schematically illustrates a cross-sectional view of another example apparatus 800 having an electromagnet 820 configured to generate a controllably varied magnetic field in accordance with certain implementations described herein. The apparatus 800 comprises a housing 810 having an external surface 812 configured to be placed on a non-magnetic material (e.g., tissue 330) overlaying a first device (e.g., implanted component 310) comprising a first ferromagnetic or ferrimagnetic material 316. The apparatus 800 further comprises a cavity 910 within the housing 810 and a permanent magnet 920 within the cavity 910, the permanent magnet 920 configured to move within the cavity 910 along a longitudinal axis 840 substantially perpendicular to the external surface 812, the permanent magnet 920 configured to generate an attractive magnetic force 922 with the first device. The apparatus 800 further comprises an electromagnet 820 within the housing 810 and configured to generate and controllably adjust a retention attractive magnetic force 930 between the electromagnet 820 and the permanent magnet 920. For example, the retention attractive magnetic force 930 can be controllably adjusted by controllably varying an electrical current flowing through the electromagnet 820.

In certain implementations, the cavity 910 containing the permanent magnet 920 comprises a first region 912 in which the permanent magnet 920 is held by the retention attractive magnetic force 930 and a second region 914 into which the permanent magnet 920 is configured to move in response to the attractive magnetic force 922 between the permanent magnet 920 and the implantable component 310 being greater than the retention attractive magnetic force 930. In certain implementations, the direction of the retention attractive magnetic force 930 is substantially opposite to the direction of the attractive magnetic force 922 applied to the permanent magnet 920 by the implantable component 310 and a magnitude of the retention attractive magnetic force 930 is controllably varied (e.g., while a direction of the retention attractive magnetic force 930 is substantially unvarying).

In certain implementations, the apparatus 800 of FIG. 9 further comprises a partition 950 within the housing 810, the partition 950 comprising a non-magnetic material and at least partially bounding the first region 912 and/or the cavity 910. The partition 950 is between the electromagnet 820 and the permanent magnet 920 (e.g., the electromagnet 820 is outside the cavity 910). The electromagnet 820 is in magnetic communication with the permanent magnet 920 to generate the retention attractive magnetic force 930 on the permanent magnet 920.

In an example use of the apparatus 800 of FIG. 9 in conjunction with an implantable component 310 beneath the recipient's tissue 330, the apparatus 800 can be placed on the recipient's tissue 330 with the permanent magnet 920 in the first region 912 of the cavity 910 and the electromagnet 820 generating a magnetic field such that the retention attractive magnetic force 930 on the permanent magnet 920 is greater than the attractive magnetic force 922 applied to the permanent magnet 920 by the magnetic interaction with the at least one internal magnetic material 316. Initially, as the electromagnet 820 is controllably varied so as to reduce the retention attractive magnetic force 930, the permanent magnet 920 remains in the first region 912. At a magnetic field at which the attractive magnetic force 922 becomes larger than the retention attractive magnetic force 930, the permanent magnet 920 moves from the first region 912 to the second region 914 of the cavity 910, thereby signaling that the attractive magnetic force 922 is greater than the retention attractive magnetic force 930. The permanent magnet 920 of certain implementations is configured to return to the first region 912 upon the attractive magnetic force 922 being less than the retention attractive magnetic force 930.

In an alternative example use of the apparatus 800 of FIG. 9, the apparatus 800 can be placed on the recipient's tissue 330 with the permanent magnet 920 in the second region 914 of the cavity 910 and the electromagnet 820 generating a magnetic field such that the retention attractive magnetic force 930 on the permanent magnet 920 is less than the attractive magnetic force 922 applied to the permanent magnet 920 by the magnetic interaction with the at least one internal magnetic material 316. Initially, as the electromagnet 820 is controllably varied so as to increase the retention attractive magnetic force 930, the permanent magnet 920 remains in the second region 914. At a magnetic field at which the attractive magnetic force 922 becomes less than the retention attractive magnetic force 930, the permanent magnet 920 moves from the second region 914 to the first region 912 of the cavity 910, thereby signaling that the attractive magnetic force 922 is less than the retention attractive magnetic force 930. In certain such example uses, the initial retention attractive magnetic force 930 is sufficiently large that a recipient having a relatively thin SFT does not experience a painful amount of pressure due to the attractive magnetic force 922 between the implantable component 310 and the permanent magnet 920.

In certain implementations, the movement of the permanent magnet 920 from the first region 912 to the second region 914, or vice versa, causes an audible sound (e.g., click) due to the permanent magnet 920 hitting a portion of the cavity 910. For example, the first region 912 and/or the second region 914 can comprise a metallic plate or other component configured to increase the sound of the permanent magnet 920 hitting the first region 912 and/or the second region 914. In certain other implementations, the housing 810 comprises a substantially optically transparent portion (e.g., window; opening) configured to allow a user to visually see the position of the permanent magnet 920 (e.g., whether the permanent magnet 920 is in the first region 912 or the second region 914).

In still other implementations, the apparatus 800 comprises at least one sensor (e.g., optical sensor; infrared sensor; electrical sensor; magnetic sensor; mechanical switch) configured to generate a sensor signal indicative of the position of the permanent magnet 920 relative to the cavity 910 (e.g., the at least one sensor comprising the permanent magnet 920), at least one signal processor (e.g., microcontroller) configured to generate a display control signal in response to the sensor signal, and at least one indicator (e.g., LED light; alphanumeric display device; sound speaker) configured to present a user-receivable signal (e.g., light; pattern; alphanumeric character; sound) indicative of the position of the permanent magnet 920 relative to the cavity 910 (e.g., to indicate that the permanent magnet 920 has moved to/from the first region 912 and/or the second region 914). In certain other implementations, instead of comprising at least one display device and at least one signal processor, the apparatus 800 is configured to wirelessly transmit the sensor signal to a separate device comprising at least one display device.

In certain implementations, the display device (e.g., of the apparatus 800 or of a separate device) is configured to provide information regarding the SFT of the recipient's tissue 330 and/or a suggested magnetic configuration for an external component 320 (e.g., the suggested magnetic configuration selected from among a finite set of magnetic configurations available for the external component 320). For example, the information can be generated in response to the retention attractive magnetic force 930 at which the position of the permanent magnet 920 changes from the first region 912 to the second region 914 or vice versa (e.g., using a predetermined relationship between the magnetic strength of the at least one internal magnetic material 316 and the strength of the retention attractive magnetic force 930).

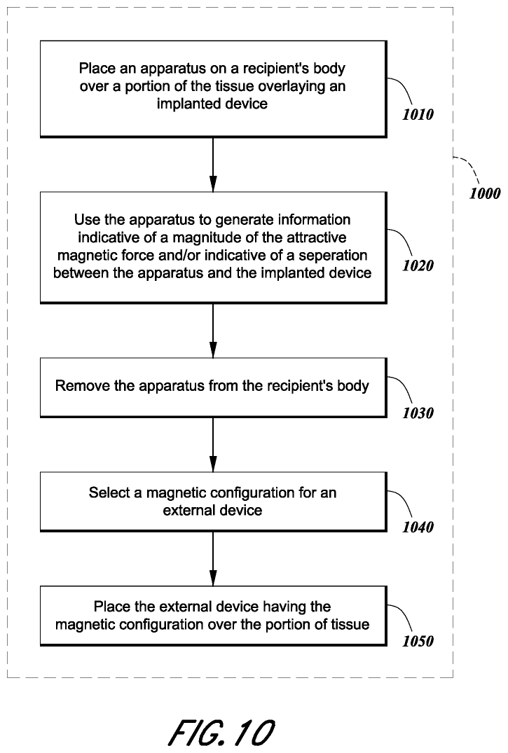

FIG. 10 is a flow diagram of an example method 1000 in accordance with certain implementations described herein. While the method 1000 is described by referring to some of the structures of the example apparatus 400, 600, 800 of FIGS. 2, 3A-3E, 4, 5, 6, 7A-7B, 8, and 9, other apparatus and systems with other configurations of components can also be used to perform the method 1000 in accordance with certain implementations described herein. In certain implementations, the method 1000 is performed using an apparatus 400, 600, 800 that does not have the functionality of an external device that is a part of, or operates in conjunction with, the implanted component 310 during operation of the implanted component 310.

In an operational block 1010, the method 1000 comprises placing an apparatus 400, 600, 800 on a recipient's body over a portion of tissue 330 overlaying an implanted device (e.g., implantable component 310), the apparatus 400, 600, 800 comprising a ferromagnetic or ferrimagnetic material (e.g., second ferromagnetic or ferrimagnetic material 420, 620) and/or a magnet (e.g., electromagnet 820) configured to generate an attractive magnetic force 422, 622, 822 with the implanted device.

In an operational block 1020, the method 1000 further comprises, while the apparatus 400, 600, 800 is on the recipient's body, using the apparatus 400, 600, 800 to generate information indicative of a magnitude of the attractive magnetic force 422, 622, 822 and/or indicative of a separation between the apparatus 400, 600, 800 and the implanted device. For example (see, e.g., FIGS. 2, 3A-3E, and 4), using the apparatus 400 to generate information can comprise adjusting a distance of the ferromagnetic or ferrimagnetic material (e.g., second ferromagnetic or ferrimagnetic material 420) from the implanted device to determine a distance corresponding to sufficient attractive magnetic force to hold the external device on the recipient's body. For another example (see, e.g., FIGS. 5, 6, and 7A-7B), using the apparatus 600 to generate information comprises using a gauge 630 (e.g., comprising a rotatable portion 632; comprising at least one elastically deflectable element 650) of the apparatus 600. For still another example, (see, e.g., FIGS. 8 and 9), using the apparatus 800 to generate information can comprise adjusting a magnetic strength of the electromagnet 820 to determine a magnetic strength corresponding to sufficient attractive magnetic force to hold the external device on the recipient's body. In certain implementations, the method 1000 further comprises using the information to estimate a thickness of the portion of tissue 330 between the implanted device and the housing 410, 610, 810 of the apparatus 400, 600, 800.

In an operational block 1030, the method 1000 further comprises removing the apparatus 400, 600, 800 from the recipient's body. In an operational block 1040, the method 1000 further comprises, in response to the information, selecting a magnetic configuration for an external device (e.g., external device 320). For example, the external device can be configured to wirelessly communicate power and/or data with the implanted device (e.g., to wirelessly transmit power and/or data to the implanted device; to wirelessly receive data from the implanted device). In an operational block 1050, the method 1000 further comprises placing the external device having the selected magnetic configuration over the portion of tissue 330 such that the external device is magnetically held on the recipient's body. For example, the method 1000 can further comprise placing the external device in wireless communication with the implanted device.

For example, selecting the magnetic configuration can comprise selecting at least one second magnet from a finite set of magnets compatible for being placed within the external device, and placing the external device having the selected magnetic configuration over the portion of tissue can comprise placing the external device with the at least one second magnet over the portion of tissue such that the at least one second magnet magnetically holds the external device on the recipient's body. For another example, selecting the magnetic configuration can comprise selecting a distance of at least one second magnet within the external device from an outside surface of the external device, and placing the external device having the selected magnetic configuration over the portion of tissue can comprise placing the external device with the outside surface on the portion of tissue such that the at least one second magnet magnetically holds the external device on the recipient's body.

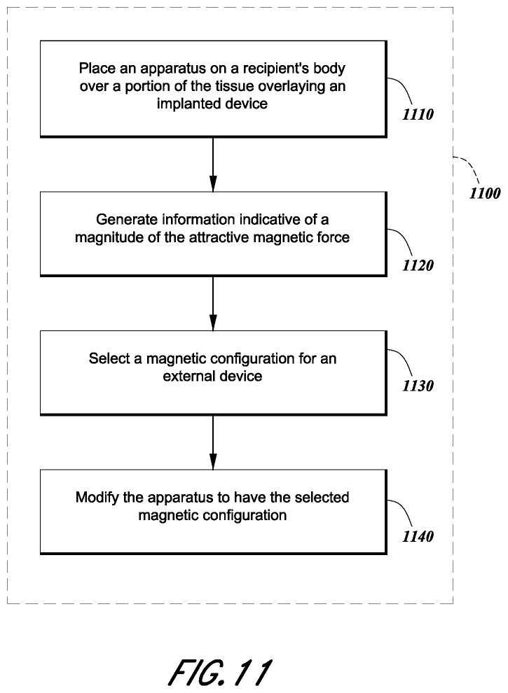

FIG. 11 is a flow diagram of an example method 1100 in accordance with certain implementations described herein. While the method 1100 is described by referring to some of the structures of the example apparatus 600 of FIG. 5, other apparatus and systems with other configurations of components can also be used to perform the method 1100 in accordance with certain implementations described herein.

In an operational block 1110, the method 1100 comprises placing an apparatus 600 on a recipient's body over a portion of tissue 330 overlaying an implanted device (e.g., implantable component 310). In certain implementations, the apparatus 600 has the functionality of an external device that is a part of, or operates in conjunction with, the implanted device during operation of the implanted device. For example, the apparatus 600 can be configured to wirelessly communicate power and/or data with the implanted device (e.g., to wirelessly transmit power and/or data to the implanted device; to wirelessly receive data from the implanted device). The apparatus 600 can comprise a permanent magnet (e.g., second ferromagnetic or ferrimagnetic material 620) configured to generate an attractive magnetic force 622 with the implanted device.

In an operational block 1120, the method 1100 further comprises, while the apparatus 600 is on the recipient's body, generating information indicative of a magnitude of the attractive magnetic force 622 and/or indicative of a separation distance between the apparatus 600 and the implanted device. In certain implementations, the method 1100 further comprises using the information to estimate a thickness of the portion of tissue 330 between the implanted device and the housing 610 of the apparatus 600.

For example, said generating information comprises using a gauge 630 of the apparatus 600 (see, e.g., FIG. 5), the gauge 630 configured to detect the attractive magnetic force 622 retaining the apparatus 600 on the recipient's body (e.g., during operation of the implanted device). The gauge 630 can generate a signal indicative of the information (e.g., measured pressure and/or force) and can communicate the signal to circuitry of the apparatus 600 and/or to circuitry of a display device (e.g., smartphone; smart tablet; computer) separate from the apparatus 600. For another example, said generating information can comprise using a pressure sensor separable from the apparatus 600. For example, the pressure sensor can be a sheet-style pressure sensor having a pressure-sensing region configured to be placed between the apparatus 600 and the tissue 330, the pressure sensor having features to align the pressure-sensing region with the implanted device and/or the apparatus 600. The pressure sensor can further comprise a communication interface (e.g., wired; wireless) configured to transmit signals indicative of the detected pressure to circuitry of the apparatus 600 and/or to circuitry of a display device separate from the apparatus 600 (e.g., circuitry of a computer configured for programming the apparatus 600).

In an operational block 1130, the method 1100 further comprises, in response to the information, selecting (e.g., recommending) a magnetic configuration for the apparatus 600 (e.g., external sound processor). For example, said selecting the magnetic configuration for the apparatus 600 can be performed by the circuitry of the apparatus 600, circuitry of the display device separate from the apparatus 600, or by a user that receives the information (e.g., from a display of the apparatus 600 or of the display device separate from and in communication with the circuitry of the apparatus 600).

In an operational block 1140, the method 1100 further comprises modifying the apparatus 600 to have the selected magnetic configuration. For example, said selecting the magnetic configuration can comprise selecting at least one second magnet from a finite set of magnets (e.g., permanent magnets) compatible for being placed within the apparatus 600, and said modifying can comprise removing the permanent magnet from the apparatus 600 (e.g., after removing the apparatus 600 from over the tissue 330) and inserting the selected at least one second magnet into the apparatus 600 (e.g., then replacing the apparatus 600 with the selected magnetic configuration over the tissue 330, such that the selected at least one second magnet magnetically holds the apparatus 600 on the recipient's body and the apparatus 600 is in wireless communication with the implanted device). For another example, said selecting the magnetic configuration can comprise selecting a distance of the permanent magnet within the apparatus 600 from an outside surface of the apparatus 600 (e.g., the surface in contact with the tissue 330), and said modifying the apparatus 600 can comprise moving the permanent magnet to have the selected distance from the outside surface of the apparatus 600. In certain implementations, moving the permanent magnet is performed while the apparatus 600 remains over the tissue 330 (e.g., remains in wireless communication with the implanted device), while in certain other implementations, moving the permanent magnet is performed after removing the apparatus 600 from over the tissue 330 (e.g., such that the apparatus 600 is not in wireless communication with the implanted device) and before replacing the apparatus 600 having the selected magnetic configuration over the tissue 330 such that the permanent magnet again magnetically holds the apparatus 600 on the recipient's body (e.g., and is again in wireless communication with the implanted device).

In certain implementations the apparatus 600 comprises a sensor configured to detect the existing magnetic configuration of the apparatus 600. For example, the permanent magnet of the apparatus 600 can have at least one property (e.g., electrical; magnetic), structure (e.g., shape; recess; protrusion) and/or appearance (e.g., color; alphanumeric characters; barcode) indicative of the magnet strength and/or magnet type of the permanent magnet existing in the apparatus 600. The sensor (e.g., optical sensor; infrared sensor; electrical sensor; magnetic sensor; mechanical switch) can be configured to detect the property, structure, and/or appearance, to generate a signal indicative of the corresponding magnet strength and/or magnet type of the permanent magnet existing in the apparatus 600, and to communicate the signal to the circuitry of the apparatus 600 or of the display device separate from the apparatus 600. The circuitry can be configured to perform said selecting a magnetic configuration for the apparatus 600 in the operational block 1130 based, at least in part, on the signal from the sensor. Alternatively, the circuitry can be configured to communicate a recommendation regarding selected magnet strength and/or magnet type of the permanent magnet to the user for the user to consider in selecting the magnetic configuration and/or in deciding whether to use a headband or other device to provide additional retention force to hold the apparatus 600 on the recipient's body.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of various devices, various implementations described herein can be incorporated in a variety of other suitable devices, methods, and contexts. More generally, as can be appreciated, certain implementations described herein can be used in a variety of implantable medical device contexts that can benefit from certain attributes described herein.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein but should be defined only in accordance with the claims and their equivalents.

1. An apparatus comprising:
a housing having an external surface configured to be placed on a non-magnetic material overlaying a first device comprising a first ferromagnetic or ferrimagnetic material;
a second ferromagnetic or ferrimagnetic material within the housing, the second ferromagnetic or ferrimagnetic material configured to generate an attractive magnetic force with the first device; and
a movable coupler in mechanical communication with the housing and the second ferromagnetic or ferrimagnetic material, the coupler configured to controllably adjust a distance between the second ferromagnetic or ferrimagnetic material and the first device along a longitudinal axis substantially perpendicular to the surface.

2. The apparatus of claim 1, wherein the first device comprises a medical implant and the non-magnetic material comprises tissue of the recipient.

3. The apparatus of claim 2, wherein the medical implant comprises an implanted portion of an auditory prosthesis system, the apparatus is configured to approximate a mass, size, and/or shape of an external portion of the auditory prosthesis system magnetically coupled to the implanted portion.

4. The apparatus of claim 3, wherein the attractive magnetic force is configured to approximate a magnetic force between the implanted portion and the external portion.

5. The apparatus of claim 1, wherein the first device has a first dipole magnetic moment and the second ferromagnetic or ferrimagnetic material has a second dipole magnetic moment, the second ferromagnetic or ferrimagnetic material configured to rotate within the housing such that the second dipole magnetic moment is aligned substantially parallel to the first dipole magnetic moment.

6. The apparatus of claim 5, wherein the second ferromagnetic or ferrimagnetic material has a substantially spherical shape.

7. The apparatus of claim 5, wherein the second ferromagnetic or ferrimagnetic material comprises a visual indication of an orientation of the second dipole magnetic moment and the apparatus further comprises an optically transparent portion configured to allow a user to view the visual indication.

8. The apparatus of claim 1, wherein the coupler comprises a receptacle configured to hold the second ferromagnetic or ferrimagnetic material and a screw thread spiraled around the longitudinal axis, the screw thread configured to mate with a corresponding screw thread of the housing, the coupler configured to controllably move the second ferromagnetic or ferrimagnetic material along the direction by rotating about the longitudinal axis.

9. The apparatus of claim 1, wherein the coupler is configured to be controllably and linearly slid along the longitudinal axis, the coupler comprising a cavity containing the second ferromagnetic or ferrimagnetic material, the cavity comprising a first region in which the second ferromagnetic or ferrimagnetic material is held by a retention force and a second region into which the second ferromagnetic or ferrimagnetic material is configured to move in response to the attractive magnetic force being greater than the retention force.

10. The apparatus of claim 9, wherein the coupler further comprises a fixed magnet in magnetic communication with the second ferromagnetic or ferrimagnetic material to generate the retention force.

11. The apparatus of claim 10, wherein the coupler further comprises a non-magnetic partition between the fixed magnet and the second ferromagnetic or ferrimagnetic material.

12. The apparatus of claim 9, wherein the coupler further comprises a spring in mechanical communication with the second ferromagnetic or ferrimagnetic material to generate the retention force.

13. The apparatus of claim 9, wherein the coupler further comprises an adhesive material or a hook-and-loop fastener between the second ferromagnetic or ferrimagnetic material and an internal surface of the coupler, the adhesive material or the hook-and-loop fastener configured to generate the retention force.

14. The apparatus of claim 9, wherein the second ferromagnetic or ferrimagnetic material is configured to return to the first region upon the attractive magnetic force being less than the retention force.

15. The apparatus of claim 9, further comprising a sensor configured to detect an orientation of the apparatus relative to the Earth.

16. The apparatus of claim 1, wherein at least a portion of the housing and/or at least a portion of the coupler is substantially optically transparent such that the second ferromagnetic or ferrimagnetic material can be viewed by a user.

17. The apparatus of claim 1, wherein the coupler further comprises at least one indicator of a position and/or orientation of the coupler relative to the housing.

18. The apparatus of claim 1, wherein the coupler is configured to have a finite set of discrete positions and/or orientations relative to the housing, the finite set of discrete positions and/or orientations corresponding to attractive magnetic forces that correspond to a finite set of magnetic attractions generated by a second device having a finite set of magnetic configurations upon the second device being selectively placed at a location on the non-magnetic material overlaying the first device.

19-54. (canceled)
